(19) United States
(12) Patent Application Publication
(10) Pub. No.: US 2013/0226307 A1
(43) Pub. Date: Aug. 29, 2013

(54) COATED ENDOPROSTHESES AND RELATED SYSTEMS & METHODS

(71) Applicant: Amedica Corporation, (US)

(72) Inventor: Bryan J. McEntire, Sandy, US (US)

(73) Assignee: Amedica Corporation, Salt Lake City, UT (US)

(21) Appl. No.: 13/779,290

(22) Filed: Feb. 27, 2013

Related U.S. Application Data
(60) Provisional application No. 61/604,395, filed on Feb. 28, 2012.

(51) Int. Cl. A61F 2/32

(52) U.S. Cl. CPC A61F 2/32
USPC 623/22.4; 623/18.11; 623/23.43; 427/2.24

(57) ABSTRACT

Methods for performing a hemiarthroplasty procedure. In some implementations, the method may comprise providing a component of an endoprosthesis, such as a femoral component of a hip prosthesis. The component may comprise a silicon nitride ceramic material, and may further comprise a coated articulating surface. The coated articulating surface may comprise a coating configured to reduce a coefficient of friction of the articulating surface. The method may further comprise positioning the endoprosthesis such that the coated articulating surface is positioned adjacent to a patient's native articular cartilage. In this manner, the coated articulating surface may articulate with the native articular cartilage following the procedure.

### RELATED APPLICATIONS

This application claims the benefit under 35 U.S.C. §119(e) of U.S. Provisional Patent Application No. 61/604,395 filed Feb. 28, 2012 and titled “COATED ENDOPROSTHESES AND RELATED SYSTEMS & METHODS,” which application is incorporated herein by reference in its entirety.

### TECHNICAL FIELD

The present disclosure relates generally to implantable prostheses and, more specifically, but not exclusively, to implantable ceramic endoprostheses having coated articulation surfaces.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 illustrates a cross sectional view of an exemplary hip prosthesis in an installed position affixed to a patient's femur and acetabulum consistent with embodiments of the present disclosure;

FIG. 2 illustrates an exploded view of the hip prosthesis of FIG. 1; and

FIG. 3 is a flow chart illustrating an exemplary method consistent with embodiments of the present disclosure.

### DETAILED DESCRIPTION

Embodiments described herein may be best understood by reference to the drawings, wherein like parts are designated by like numerals throughout. It will be readily understood that the components of the present disclosure, as generally described and illustrated in the drawings herein, could be arranged and designed in a wide variety of different configurations. Thus, the following more detailed description of the embodiments of the apparatus is not intended to limit the scope of the disclosure, but is merely representative of possible embodiments of the disclosure. In some cases, well-known structures, materials, or operations are not shown or described in detail.

Pyrolytic carbon implants have, in several studies, demonstrated improved outcomes in certain regards relative to metal alloy implants. However, the typical flexural strength and fracture toughness for pyrolytic carbon are less than ideal for many applications. The typical flexural strength and fracture toughness values for pyrolytic carbon are 490 MPa and 1.67 MPa·m1/2, respectively. By contrast, silicon nitride typically has flexural strength and fracture toughness values of 1,000 MPa and 7-10 MPa·m1/2, respectively. Pyrolytic carbon also has been reported to have a coefficient of friction of about 0.15. However, the coefficient of friction can be reduced substantially by applying one or more of the various coatings disclosed herein. For example, by using a coating of diamond-like carbon (DLC), a much lower coefficient of friction can be achieved, likely on the order of 0.01 or less. Thus, in some embodiments, by combining the increased strength and toughness of a ceramic implant with the reduced coefficient of friction of DLC or another similar coating applied to the articulation surface, orthopedic implants can be created that last longer and also better preserve the life of opposing articular cartilage.

Accordingly, in an effort to improve upon one or more of the deficiencies of the prior art, including but not limited to the deficiencies discussed above, disclosed herein are embodiments of endoprostheses having one or more coatings that improve the durability, performance, or other characteristics of the implant. In some embodiments, the implant may comprise an endoprosthesis that is useful in hemiarthroplasty applications, such as unipolar artificial joints, including unipolar hip joints or surface replacements, knee joints, shoulder joints, elbow joints, spinal facet segments, ankle, carpal, metacarpal, or any other such joint with articular cartilage. In some embodiments, the implant is made up of a biocompatible ceramic. In such embodiments, one or more abrasion-resistant coatings may be applied to the ceramic endoprosthesis to, for example, improve the characteristics of articulation with a patient's native cartilage. The coating(s) may be comprised of suitable materials and compositions, as discussed in greater detail below, that are particularly configured and suited for interfacing with and preserving native articular cartilage. Thus, in some preferred embodiments, a ceramic implant may be provided that possesses an adherent coating for use as an implantable endoprosthesis that possesses high strength and toughness, excellent biocompatibility, corrosion resistance, and/or a hydrophobic articulation surface with a relatively low coefficient of friction.

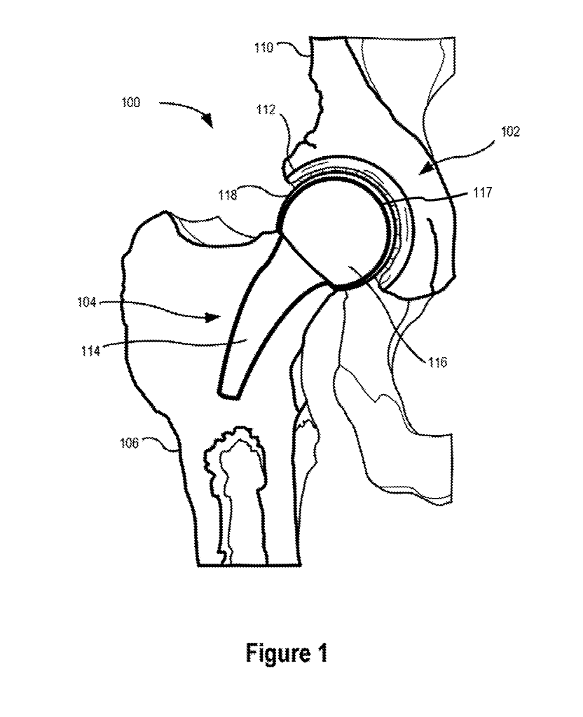

FIG. 1 illustrates a cross sectional view of an exemplary unipolar hip joint prosthesis 100 comprising a femoral component 104 having an articulating surface 117 having a coating 118. The articulating surface 117 is positioned and configured to articulate within the acetabulum 102 of a patient's pelvis 110. As shown in the figure, the acetabulum 102 includes cartilage 112. While embodiments are discussed herein in the context of an exemplary hip joint prosthesis 100, the disclosed embodiments may also be implemented in any type of implantable articulating prostheses having any number of articulating components. For example, it is contemplated that the coated ceramic endoprostheses disclosed herein could be used in connection with knee joints, shoulder joints, elbow joints, spinal joints, such as spinal facet segments, ankle joints, carpal, metacarpal, and phalangeal joints, and any other joints with articular cartilage. It is also contemplated that the coatings disclosed herein may be useful in certain non-articulating prostheses. Various embodiments disclosed herein may also have value in other industrial applications, such as engine pistons, valve train components, industrial wrist pins, knives, scissors and other cutting tools, dies, punches, watch casings, razor blades, and bearings.

The acetabulum 102 and, more particularly, the cartilage 112 of the acetabulum 102, may be configured to interface with femoral component 104 including a ball-shaped femoral head 116 configured to seat within the acetabulum 102, thereby allowing the femoral component 104 to articulate in one or more directions relative to the acetabulum 102. The femoral component 104 may further include an elongated stem 114 configured to seat and/or be affixed to an upper end of a patient's femur 106. In certain embodiments, the ball-shaped femoral head 116 and the elongated stem 114 may be integral components. In other embodiments, the ball-shaped femoral head 116 and the elongated stem 114 may be selectively detachable from one another, or may otherwise comprise modular components utilizing, for example, a threaded mechanism or another mechanism for selective detachment.

In certain embodiments, the femoral component 104 may be formed, in whole or in part, of a relatively hard and high strength biocompatible ceramic material, such as alumina, zirconia, zirconia toughed alumina, or the like. In some embodiments, the femoral component 104, or at least preferably a portion of the femoral component 104 near the articulating surface of the femoral head 116, may be made up of silicon nitride (Si3N4). In certain embodiments, the ceramic material may comprise a doped silicon nitride having relatively high hardness, tensile strength, elastic modulus, lubricity, and fracture toughness properties. Examples of suitable silicon nitride materials are described, for example, in U.S. Pat. No. 6,881,229, which is hereby incorporated by reference in its entirety. Other suitable ceramic materials may include combinations of alumina and zirconia, and/or other alumina-matrix composites. In certain embodiments, the flexural strength of the ceramic materials may range from approximately 400 MPa (e.g., for alumina) to approximately 1300 MPa (e.g., for zirconia).

In some embodiments, at least a portion of the femoral component 104 may include relatively porous ceramic bone ingrowth surfaces for secure affixation to a patient's femur 106 or other bone structure. For example, in certain embodiments, the femoral component 104 may include a portion having a porous ceramic bone ingrowth configured to be securely affixed to a portion of a patient's femur 106. In some embodiments, the femoral component 104 may include a plurality of different regions of differing porosities to respectively mimic natural cortical and cancellous bone structure. In certain embodiments, the higher porosity region may be designed to allow for improved bone ingrowth to provide for more secure and stable affixation of the implant to a patient's femur 106 or another similar structure.

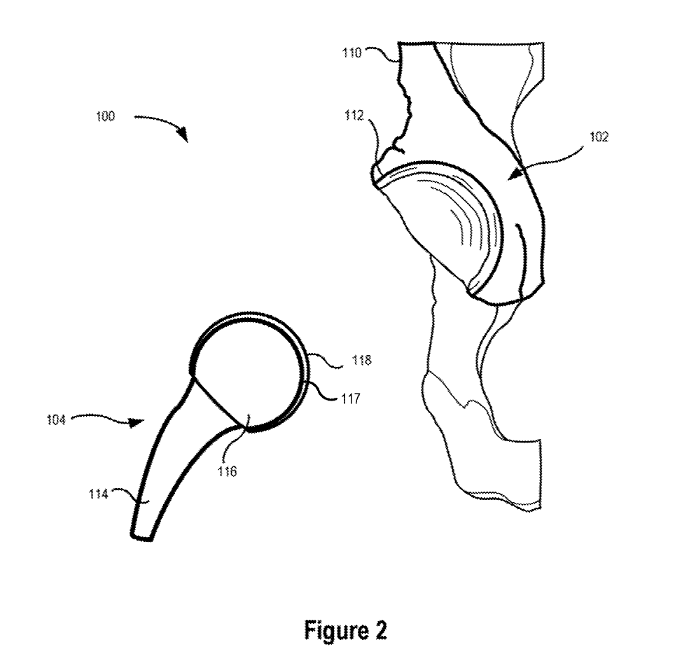

FIG. 2 illustrates an exploded view of the exemplary hip prosthesis 100 described above in reference to FIG. 1. As illustrated, the exemplary hip prosthesis 100 may comprise a femoral component 104 formed of a relatively hard and high strength biocompatible ceramic material. The femoral component 104 may include a ball-shaped femoral head 116 configured to seat within a patient's acetabulum 102 and an elongated stem 114 that may be integral with, or selectively detachable from, the ball-shaped femoral head 116.

The articulating interface surface 117 of the femoral component 104 may include one or more coatings 118. The coating(s) 118 is preferably configured to provide a highly polished articulation surface. In certain preferred embodiments, the coating may be configured and particularly suited for interfacing with a patient's native cartilage. In some embodiments, the coating may be configured to reduce wear and facilitate preservation of a patient's native cartilage. It is preferred that one or more of the materials/ingredients in the coating(s) be biocompatible, hard, lubricious, and/or abrasion resistant. Examples of suitable or potentially suitable coating materials include silicon carbide (SiC), titanium nitride (TiN), titanium diboride (TiB2), and diamond-like carbon (DLC). Preliminary tests suggest that DLC may provide superior results relative to other coatings for certain uses and implementations. Ceramic endoprostheses coated with one or more of these materials, and in particular coatings including DLC, appear to possess desired strength, toughness, biocompatibility, and/or corrosion resistance relative to other endoprostheses. In addition, when used as an articulation surface, the coatings described herein may provide a hydrophobic surface with a reduced coefficient of friction.

Various methods may be used in order to apply the coatings disclosed herein to a surface of an endoprosthesis, such as the articulation surface of such an implant. For example, coatings may be applied by way of a variety of processes known by those skilled in the art. Broadly, these may include, for example, physical vapor deposition (PVD) or chemical vapor deposition (CVD) processes, but, more specifically, can be low or high-temperature reactive CVD (i.e., LT-CVD, HT-CVD), DC or RF plasma-assisted CVD, DC or RF assisted PVD, balanced or unbalanced magnetron sputtering, ion-beam assisted deposition (IBAD), filtered cathodic arc deposition (FCAD), pulsed laser ablation and deposition (PLAD), electron cyclotron resonance CVD (ECR-CVD), or any other appropriate deposition method physical vapor deposition (PVD) or chemical vapor deposition (CVD) processes.

In some embodiments, by applying a suitable coating, such as a DLC coating, to a suitably strong and tough endoprosthesis substrate, such as a silicon nitride ceramic, an implant may be created that is particularly suited for use in hemiarthroplasty procedures. More particularly, such embodiments may have a desired combination of attributes, such as high flexural strength, high fracture toughness, high hardness, a highly wear-resistant surface, and an ultra-low friction coefficient, such as one on the order of about 0.01 or less. Moreover, when such embodiments are used in connection with hemiarthroplasty procedures, they may provide improved performance and extended in-vivo longevity against native articular cartilage.

Applying the coating 118 directly to the articulating surface 117 of the femoral component 104 may ensure good adhesion to the ceramic substrate. In certain embodiments, this adhesion may be due to covalent bonding between the coating 118 and the ceramic articulating interface surface(s) 117 of the femoral component 104. For example, in embodiments in which the substrate is a silicon nitride ceramic, and the coating applied to the substrate is or includes DLC, a covalent bond may form between the silicon atoms in the substrate and the carbon atoms in the coating.

In certain embodiments, applying one or more coatings to one or more ceramic articulating surfaces of an endoprosthesis may, in addition to one or more of the benefits discussed above, increase the surface hardness of the articulating interface surface(s). For example, a DLC coating applied using a PVD process in some embodiments may result in a surface hardness ranging from approximately 20 GPa to 40 GPa. A SiC coating applied using a CVD process may result in a surface hardness of approximately 27 GPa. Applying one or more coatings to the articulating surface may also reduce undesirable wearing of the articulating interface surfaces and facilitate preservation of opposing native articular cartilage, as discussed above.

As also discussed above, the coating 118 may also reduce the coefficient of friction at the articulating interface surface(s) 117 of the femoral head 116. In some embodiments, reducing the coefficient of friction at the articulating surface of the femoral head 116, or another similar articulating surface of a different endoprosthesis, may result in less or fewer audible noises being produced by the prosthesis during use. This is particularly true in embodiments configured for use in full-hip replacements or other non-hemiarthroplasty procedures. For example, in certain embodiments, an uncoated ceramic articulating interface surface may have a coefficient of friction ranging from approximately 0.1 to 0.4, whereas a coated articulating interface surface (e.g., coated using DLC) may have a coefficient of friction of about 0.005 to about 0.05. In some such embodiments, the coefficient of friction may be on the order of about 0.008 to about 0.03. In some such embodiments, the coefficient of friction may be on the order of about 0.009 to about 0.02. In some such embodiments, the coefficient of friction may be on the order of about 0.01. Some embodiments may comprise a coating having a coefficient of friction of less than about 0.01.

In some embodiments, only the articulating surface or surfaces of the implant may be coated. For example, in embodiments comprising a femoral component having an articulating surface configured to articulate within the acetabulum of a patient's pelvis, only the articulating surface may be coated. More particularly, in such embodiments, the stem component may be uncoated, or may comprise a different coating since reduction of the coefficient of friction of such components may not be needed or desirable. For example, with reference again to FIG. 1, elongated stem 114 that is configured to seat and/or be affixed to an upper end of a patient's femur 106 may be uncoated. Ball-shaped femoral head 116, or at least the portion of femoral head 116 that is configured to articulate within cartilage 112 of acetabulum 102, may be coated, as described above.

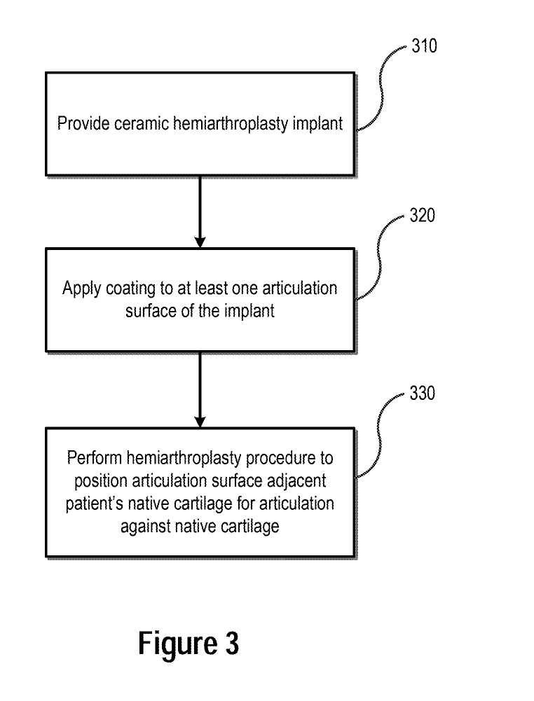

FIG. 3 is a flow chart illustrating an exemplary method accordingly to one implementation of the invention. In step 310, a ceramic hemiarthroplasty implant is provided. As discussed above, in other implementations an implant made up of an alternative material(s) and or configured for use in a non-hemiarthroplasty procedure may be provided. However, in a preferred implementation, the implant is made up at least in part of a ceramic, such as silicon nitride. Similarly, in a preferred implementation, the implant is specifically configured for use in a hemiarthroplasty procedure such that it includes an articulation surface that will eventually be placed adjacent to a patient's natural cartilage, such as the cartilage in an acetabulum.

In step 320, a coating is applied to one or more articulation surfaces of the implant. As described above, a preferred coating is DLC, and may be applied by way of, for example, a PVD or CVD process. Of course, in other implementations, other coatings may be used, as disclosed elsewhere herein. In step 330, a hemiarthroplasty procedure is performed using the coated implant. The procedure is performed such that the coated articulation surface is placed adjacent to a patient's native articular cartilage, such as the articular cartilage in the patient's acetabulum.

It will be understood by those having skill in the art that changes may be made to the details of the above-described embodiments without departing from the underlying principles presented herein. For example, any suitable combination of various embodiments, or the features thereof, is contemplated.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Throughout this specification, any reference to “one embodiment,” “an embodiment,” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, inventive aspects lie in a combination of fewer than all features of any single foregoing disclosed embodiment. It will be apparent to those having skill in the art that changes may be made to the details of the above-described embodiments without departing from the underlying principles set forth herein. The scope of the present invention should, therefore, be determined only by the following claims.

1. A method for performing a hemiarthroplasty procedure, the method comprising the steps of:
providing a component of an endoprosthesis, wherein the component comprises a silicon nitride ceramic material, and wherein the component further comprises a coated articulating surface comprising a coating, wherein the coating comprises at least one of diamond-like carbon, silicon carbide, titanium nitride, titanium diboride, titanium carbonitride, titanium aluminum nitride, chromium nitride, chromium carbonitride, and titanium silicon carbonitride; and
positioning the endoprosthesis such that the coated articulating surface is positioned adjacent to a patient's native articular cartilage such that the coated articulating surface is configured to articulate with the native articular cartilage following the procedure.

2. The method of claim 1, wherein the silicon nitride ceramic material comprises a doped silicon nitride ceramic material.

3. The method of claim 2, wherein the doped silicon nitride ceramic material comprises dopants selected from the group consisting of yttrium oxide, magnesium oxide, strontium oxide, aluminum oxide, and combinations thereof.

4. The method of claim 1, wherein the native articular cartilage comprises articular cartilage in the patient's acetabulum.

5. The method of claim 1, wherein the coated articulating surface has a coefficient of friction of between about 0.008 and about 0.03.

6. The method of claim 5, wherein the coated articulating surface has a coefficient of friction of between about 0.009 and about 0.02.

7. The method of claim 6, wherein the coated articulating surface has a coefficient of friction of about 0.01.

8. The method of claim 1, wherein the component further comprises an uncoated surface.

9. A method for applying a coating to an articulating surface of an endoprosthesis, the method comprising the steps of:
providing a component of an endoprosthesis, wherein the component comprises an articulating surface, wherein the articulating surface is configured to interface with a patient's native articular cartilage, and wherein the component comprises a silicon nitride ceramic material;
applying a coating to the articulating surface, wherein the coating comprises at least one of diamond-like carbon, silicon carbide, titanium nitride, titanium diboride, titanium carbonitride, titanium aluminum nitride, chromium nitride, chromium carbonitride, and titanium silicon carbonitride.

10. The method of claim 9, wherein the silicon nitride ceramic material comprises a doped silicon nitride ceramic material.

11. The method of claim 10, wherein the doped silicon nitride ceramic material comprises dopants selected from the group consisting of yttrium oxide, magnesium oxide, strontium oxide, aluminum oxide, and combinations thereof.

12. The method of claim 9, wherein the component comprises a femoral component, and wherein the articulating surface comprises a surface of a ball-shaped head of the femoral component.

13. The method of claim 9, wherein the step of applying a coating to the articulating surface comprises applying the coating using at least one of a physical vapor deposition and a chemical vapor deposition process.

14. The method of claim 9, wherein the step of applying a coating to the articulating surface comprises applying a diamond-like carbon material to the articulating surface.

15. The method of claim 14, wherein the step of applying a coating to the articulating surface comprises applying a diamond-like carbon material to the articulating surface using a physical vapor deposition process.

16. The method of claim 9, wherein the step of applying a coating to the articulating surface reduces the coefficient of friction of the articulating surface from between about 0.1 and about 0.4 to between about 0.005 and about 0.05.

17. The method of claim 9, wherein the step of applying a coating to the articulating surface reduces the coefficient of friction of the articulating surface to between about 0.008 and about 0.03.

18. The method of claim 17, wherein the step of applying a coating to the articulating surface reduces the coefficient of friction of the articulating surface to between about 0.009 and about 0.02.

19. The method of claim 18, wherein the step of applying a coating to the articulating surface reduces the coefficient of friction of the articulating surface to about 0.01.

20. A method for performing a hip hemiarthroplasty procedure, the method comprising the steps of:
providing a femoral component comprising a femoral head and a stem, wherein the stem is configured to be coupled with an upper end of a patient's femur, wherein the femoral component comprises a doped silicon nitride ceramic material comprising dopants selected from the group consisting of yttrium oxide, magnesium oxide, strontium oxide, aluminum oxide, and combinations thereof, wherein the femoral head comprises a coated articulating surface comprising a coating, wherein the coating comprises at least one of diamond-like carbon, silicon carbide, titanium nitride, titanium diboride, titanium carbonitride, titanium aluminum nitride, chromium nitride, chromium carbonitride, and titanium silicon carbonitride, wherein the coated articulating surface has a coefficient of friction of no more than about 0.01, and wherein the femoral component other than the coated articulating surface has a coefficient of friction of at least about 0.1; and
positioning the femoral component such that the coated articulating surface is positioned adjacent to a patient's native articular cartilage within the patient's acetabulum such that the coated articulating surface is configured to articulate with the native articular cartilage following the procedure.
